(19) United States
(12) Patent Application Publication
(10) Pub. No.: US 2015/0025283 A1
(43) Pub. Date: Jan. 22, 2015

(54) Process and Catalyst for C9+ Aromatics Conversion

(71) Applicant: ExxonMobil Chemical Patents Inc., Baytown, TX (US)

(72) Inventors: Jane C. Cheng, Bridgewater, NJ (US); Christopher G. Oliveri, Stewartsville, NJ (US)

(21) Appl. No.: 14/311,393

(22) Filed: Jun. 23, 2014

Related U.S. Application Data
(60) Provisional application No. 61/856,358, filed on Jul. 19, 2013.

(51) Int. Cl. C07C 6/06

(52) U.S. Cl. CPC C07C 6/06
USPC 585/321; 422/630; 585/486

(57) ABSTRACT

The invention is directed to a multimetallic catalyst and its use in a reactor system in a C9+ aromatics conversion process in order to reduce the saturation of aromatic species, reduce the production of C6+ non-aromatics byproducts, and achieve higher benzene purity. The multimetallic catalyst exhibits improved selectivity towards aromatic hydrocarbons in comparison to a traditional Pt/ZSM-5 catalyst and comprises ZSM-5, a Group 6-10 metal, and an additional metal not in Group 6-10. The C9+ aromatics conversion reactor system comprises a top bed containing the multimetallic catalyst for dealkylation of ethyl and propyl side chains, a second bed containing a catalyst comprising a hydrogenation component for transalkylation, and an optional third bed containing a catalyst without a hydrogenation component to convert non-aromatic hydrocarbons to gas products.

### CROSS REFERENCE TO RELATED APPLICATIONS

This application claims the benefit of priority to U.S. Provisional Application No. 61/856,358, filed Jul. 19, 2013, the disclosure of which is fully incorporated herein by reference.

### FIELD OF THE INVENTION

The invention relates to transalkylation reactions and a catalyst therefor, and more particularly for C9+ aromatics conversion.

### BACKGROUND OF THE INVENTION

The manufacture of xylene using transalkylation processes utilize one or more catalysts to convert feed streams containing benzene and/or toluene (collectively, C7-aromatic hydrocarbons) and feed streams containing heavy aromatics, i.e., C9+ aromatic hydrocarbons, into a xylene-containing product stream. The demand for xylenes, particularly paraxylene, has increased in proportion to the increase in demand for polyester fibers and film. Supplying the ever-increasing demand has required solving many problems in the production of paraxylene by transalkylation, such as discussed in U.S. Pat. Nos. 5,030,787; 5,763,720; 5,942,651; 6,893,624; 7,148,391; 7,439,204; 7,553,791; 7,663,010; 8,071,828; 8,163,966; and 8,183,424; U.S. Patent Publications 2010-0298117 and 2012-0024755; U.S. patent application Ser. No. 13/811,403; and U.S. Provisional Patent Applications 61/418,212; 61/496,262; and 61/829,360. The value of the product is so great that these processes still merit improvement and there is constant research in this area.

It is known, for instance, to provide a feedstream comprising benzene and/or toluene (“C7− aromatic hydrocarbons”) and C9+ aromatic hydrocarbon to a catalyst such as ZSM-12 comprising a hydrogenation component and a support to provide for dealkylation/transalkylation and then optionally, the product contacts a second catalyst, such as ZSM-5 without a hydrogenation component to crack certain undesired co-boilers that make separation of the desired product(s) more difficult. A second example separates the functions of dealkylation and transalkylation, passing a feedstream(s) comprising C7− and C9+ aromatic hydrocarbons to a catalyst such as ZSM-5 comprising a hydrogenation component to facilitate dealkylation, followed by contact with a catalyst such as ZSM-12 comprising a hydrogenation component for transalkylation. Optionally, a third catalyst without a hydrogenation component can be used to crack the undesired co-boilers, as in the first example.

A typical feed to such process can be any conventional C8+ aromatic hydrocarbon feed available in a petroleum or petrochemical refinery, such as a catalytic reformate, FCC or TCC naphtha, or a xylene isomerizate from which heptanes and lighter components have been removed. The feed is initially passed through a xylenes fractionation column or columns to remove the C₈ aromatic components from the feed and leave a C9+ aromatic hydrocarbon-rich fraction which can then be fed to a transalkylation reactor for reaction with benzene and/or toluene in the presence of a transalkylation catalyst system, such as described above, to produce lighter aromatic products, primarily benzene, toluene, and xylenes (collectively, “BTX”). These components can then be separated by methods well-known in the art, and all or a portion of the benzene and toluene can be recycled through the transalkylation system.

A dual metal system has recently been proposed for the isomerization of paraxylene-depleted streams. See U.S. Provisional Patent Application No. 61/604,926 [Attorney Docket Number 2012EM014]. See also International Publication WO 1996/002612 A1.

The present inventors have surprisingly discovered a dual metal system that improves the dealkylation of heavy aromatics, thus, providing an improved system for transalkylation.

### SUMMARY OF THE INVENTION

The invention is directed to a dual metal system for dealkylation of heavy aromatics, a system for transalkylation using said dual metal system, and processes for using the same.

It is an object of the invention to provide an improved transalkylation catalyst system.

It is another object of the invention to provide an improved catalyst system for conversion of streams containing C9+ aromatic hydrocarbons to streams comprising a lower concentration of heavy aromatic hydrocarbons.

It is another object of the invention to reduce aromatic ring saturation, C6+ non-aromatic byproducts, and improve benzene co-product purity, in a process for transalkylation to produce paraxylene.

These and other objects, features, and advantages will become apparent as reference is made to the following detailed description, preferred embodiments, examples, and appended claims.

### BRIEF DESCRIPTION OF THE DRAWINGS

FIGS. 1-6 show experimental results comparing catalyst systems according to the present invention with a conventional system.

### DETAILED DESCRIPTION OF THE INVENTION

According to the invention, a dual metal catalyst system for dealkylation of heavy aromatics is provided that results, in embodiments, in at least one of reduced aromatic ring saturation, reduced C6+ non-aromatic byproducts, and improved benzene purity, in a process for conversion of a feedstream comprising said heavy aromatics. A system for transalkylation using said dual metal system as a top bed, and the catalyst system are also embodiments of the invention.

The dual metal catalyst system may be used as a top bed in a system further comprising, as a second bed, a catalyst bed comprising at least one catalyst selected to facilitate transalkylation of C9+ aromatics and benzene and/or toluene to produce xylenes, and/or facilitate transalkylation of C9+ aromatics and benzene to produce toluene, which may then be further processed, such as in the same transalkylation system or a different system, to produce xylenes, or in another process, such as toluene alkylation with an alkylation agent such as methanol and/or dimethyl ether to produce xylenes, or in a toluene disproportionation system to produce benzene and xylenes, or some other system or combination thereof

### Definitions

For the purpose of this specification and appended claims, the following terms are defined. The term “Cₙ” hydrocarbon wherein n is a positive integer, e.g., 1, 2, 3, 4, or 5, means a hydrocarbon having n number of carbon atom(s) per molecule. The term “Cₙ₋” hydrocarbon wherein n is a positive integer, e.g., 1, 2, 3, 4, or 5, means hydrocarbon having at least n number of carbon atom(s) per molecule. The term “Cₙ₋” hydrocarbon wherein n is a positive integer, e.g., 1, 2, 3, 4, or 5, means hydrocarbon having no more than n number of carbon atom(s) per molecule. The term “aromatics” means hydrocarbon molecules containing at least one aromatic core. The term “hydrocarbon” encompasses mixtures of hydrocarbon, including those having different values of n. The term “syngas” means a gaseous mixture comprising hydrogen, carbon monoxide, and optionally some carbon dioxide.

As used herein, the numbering scheme for the groups of the Periodic Table of the Elements is as disclosed in Chemical and Engineering News, 63(5), 27 (1985).

### First Catalyst Bed

The first catalyst bed employed in the present catalyst system accommodates a first catalyst comprising a M/ZSM-5 catalyst, where M is a metal or compound thereof selected from at least one of Groups 6 to 10 of the Periodic Table of the Elements, and at least one additional metal not from Groups 6 to 10, selected from Groups 1 to 5 or 11 to 14. ZSM-5 is described in detail in U.S. Pat. No. 3,702,886 and Re. 29,948. In one preferred embodiment, the ZSM-5 has an average crystal size of less than 0.1 micron, such as about 0.05 micron.

Conveniently, the first molecular sieve has an alpha value in the range of about 100 to about 1500, such as about 150 to about 1000, for example about 300 to about 600. Alpha value is a measure of the cracking activity of a catalyst and is described in U.S. Pat. No. 3,354,078 and in the Journal of Catalysis, Vol. 4, p. 527 (1965); Vol. 6, p. 278 (1966); and Vol. 61, p. 395 (1980), each incorporated herein by reference as to that description. The experimental conditions of the test used herein include a constant temperature of 538° C. and a variable flow rate as described in detail in the Journal of Catalysis, Vol. 61, p. 395.

Typically, the first catalyst comprises at least 1 wt %, preferably at least 10 wt %, more preferably at least 50 wt %, and most preferably at least 65 wt %, of the first molecular sieve.

In addition to the ZSM-5, the first catalyst comprises at least one metal or compounds thereof of Groups 6 to 10 of the Periodic Table of the Elements. The first metal is generally selected from platinum, palladium, iridium, rhenium and mixtures thereof In one embodiment, the first metal comprises platinum.

The first catalyst also comprises at least one additional metal, not from Groups 6 to 10, and preferably selected from Groups 1, 2, or 11 to 14. The additional metal is conveniently selected from at least one of tin, copper, silver, calcium, and magnesium.

Conveniently, the Group 6-10 metal is present in the first catalyst in an amount between about 0.001 and about 5 wt %, preferably about 0.110 to 0.120 wt %, based on the weight of the M/ZSM-5 catalyst. The Group 6-10 metal and the additional metal not from Group 6-10 have a molar ratio between about 0.7:1 to 1.3:1, preferably about 1:1.

In most cases, the first catalyst also comprises a binder or matrix material that is resistant to the temperatures and other conditions employed in the present transalkylation process. Such materials include active and inactive materials and synthetic or naturally occurring zeolites, as well as inorganic materials such as clays, silica and/or metal oxides such as alumina The inorganic material may be either naturally occurring, or in the form of gelatinous precipitates or gels including mixtures of silica and metal oxides. Use of a binder or matrix material which itself is catalytically active, may change the conversion and/or selectivity of the catalyst composition. Inactive materials suitably serve as diluents to control the amount of conversion so that transalkylated products can be obtained in an economical and orderly manner without employing other means for controlling the rate of reaction. These catalytically active or inactive materials may include, for example, naturally occurring clays, e.g., bentonite and kaolin, to improve the crush strength of the catalyst composition under commercial operating conditions.

Naturally occurring clays that can be composited with the first molecular sieve as a binder for the catalyst composition include the montmorillonite and kaolin family, which families include the subbentonites, and the kaolins commonly known as Dixie, McNamee, Georgia and Florida clays or others in which the main mineral constituent is halloysite, kaolinite, dickite, nacrite or anauxite. Such clays can be used in the raw state as originally mined or initially subjected to calcination, acid treatment or chemical modification.

In addition to the foregoing materials, the first molecular sieve can be composited with a porous matrix binder material, such as an inorganic oxide selected from the group consisting of silica, alumina, zirconia, titania, thoria, beryllia, magnesia, and combinations thereof, such as silica-alumina, silica-magnesia, silica-zirconia, silica-thoria, silica-beryllia, silica-titania, as well as ternary compositions such as silica-alumina-thoria, silica-alumina-zirconia, silica-alumina-magnesia and silica-magnesia-zirconia. It may also be advantageous to provide at least a part of the foregoing porous matrix binder material in colloidal form so as to facilitate extrusion of the catalyst composition.

Typically the first molecular sieve is admixed with the binder or matrix material so that the first catalyst composition contains the binder or matrix material in an amount ranging from 5 to 95 wt %, and typically from 10 to 60 wt %.

### Second Catalyst Bed

The second catalyst bed accommodates a second catalyst comprising a second molecular sieve having a Constraint Index less than 3 and optionally one or more metals or compounds thereof of Groups 6 to 12 of the Periodic Table of the Elements.

Constraint Index is a convenient measure of the extent to which an aluminosilicate or other molecular sieve provides controlled access to molecules of varying sizes to its internal structure. For example, molecular sieves which provide a highly restricted access to and egress from its internal structure have a high value for the Constraint Index. Molecular sieves of this kind usually have pores of small diameter, e.g. less than 5 Angstroms. On the other hand, molecular sieves which provide relatively free access to their internal pore structure have a low value for the Constraint Index, and usually pores of large size. The method by which constraint index is determined is described fully in U.S. Pat. No. 4,016,218, which is incorporated herein by reference for the details of the method.

Suitable molecular sieves for use in the second catalyst composition comprise at least one of zeolite beta, zeolite Y, Ultrastable Y (USY), Dealuminized Y (Deal Y), mordenite, NU-87, ZSM-3, ZSM-4 (Mazzite), ZSM-12, ZSM-18, MCM-22, PSH-3, SSZ-25, MCM-36, MCM-49, MCM-56, EMM-10, EMM-10-P and ZSM-20. Zeolite ZSM-4 is described in U.S. Pat. No. 3,923,636. Zeolite ZSM-12 is described in U.S. Pat. No. 3,832,449. Zeolite ZSM-20 is described in U.S. Pat. No. 3,972,983. Zeolite Beta is described in U.S. Pat. No. 3,308,069, and Re. No. 28,341. Low sodium Ultrastable Y molecular sieve (USY) is described in U.S. Pat. Nos. 3,293,192 and 3,449,070. Dealuminized Y zeolite (Deal Y) may be prepared by the method found in U.S. Pat. No. 3,442,795. Zeolite UHP-Y is described in U.S. Pat. No. 4,401,556. Rare earth exchanged Y (REY) is described in U.S. Pat. No. 3,524,820. Mordenite is a naturally occurring material but is also available in synthetic forms, such as TEA-mordenite (i.e., synthetic mordenite prepared from a reaction mixture comprising a tetraethylammonium directing agent). TEA-mordenite is disclosed in U.S. Pat. Nos. 3,766,093 and 3,894,104. MCM-22 is described in U.S. Pat. No. 4,954,325. PSH-3 is described in U.S. Pat. No. 4,439,409. SSZ-25 is described in U.S. Pat. No. 4,826,667. MCM-36 is described in U.S. Pat. No. 5,250,277. MCM-49 is described in U.S. Pat. No. 5,236,575. MCM-56 is described in U.S. Pat. No. 5,362,697.

In one preferred embodiment, the second molecular sieve comprises ZSM-12 and especially ZSM-12 having an average crystal size of less than 0.1 micron, such as about 0.05 micron.

Conveniently, the second molecular sieve has an alpha value of at least 20, such as from about 20 to about 500, for example from about 30 to about 100.

Generally, the second molecular sieve is an aluminosilicate having a silica to alumina molar ratio of less than 500, typically from about 50 to about 300.

Typically, the second catalyst comprises at least 1 wt %, preferably at least 10 wt %, more preferably at least 50 wt %, and most preferably at least 65 wt %, of the second molecular sieve.

Optionally, the second catalyst comprises a hydrogenation component consisting of at least one and preferably at least two metals or compounds thereof of Groups 6 to 12 of the Periodic Table of the Elements. Generally, the second catalyst comprises the same first and second metals present in the same amounts as contained by the first catalyst.

Generally, the second catalyst also contains a binder or matrix material, which can be any of the materials listed as being suitable for the first catalyst and can be present in an amount ranging from 5 to 95 wt %, and typically from 10 to 60 wt %, of the second catalyst composition.

Conveniently, the weight ratio of the first catalyst to the second catalyst is in the range of 5:95 to 75:25.

### Optional Third Catalyst Bed

In addition to the first and second catalysts beds employed in the present multi-bed catalysts system, it may be desirable to incorporate a third catalyst bed downstream of the second catalyst bed and effective to crack non-aromatic cyclic hydrocarbons in the effluent from the first and second catalyst beds. The third catalyst bed accommodates a third catalyst comprising a third molecular sieve having a Constraint Index from about 1 to 12. Suitable molecular sieves for use in the third catalyst comprise at least one of ZSM-5, ZSM-11, ZSM-12, zeolite beta, ZSM-22, ZSM-23, ZSM-35, ZSM-48, ZSM-57 and ZSM-58, with ZSM-5 being preferred.

### Production of the Catalysts

The metal components of the first and second catalysts can be incorporated into the catalyst composition by co-crystallization, exchanged into the composition to the extent a Group 13 element, e.g., aluminum, is in the molecular sieve structure, impregnated therein, or mixed with the molecular sieve and binder. For example, the metal components can be impregnated in or on the molecular sieve, for example in the case of platinum, by treating the molecular sieve with a solution containing a platinum metal-containing ion. Suitable platinum compounds for impregnating the catalyst with platinum include chloroplatinic acid, platinous chloride and various compounds containing the platinum ammine complex, such as Pt(NH₃)₄Cl₂H₂O. Alternatively, a compound of the hydrogenation component may be added to the molecular sieve when it is being composited with a binder, or after the molecular sieve and binder have been formed into particles by extrusion or pelletizing. The second metal component may be incorporated into the catalyst composition at the same time and in the same manner as the first metal component. Alternatively, the second metal component may be incorporated into the catalyst composition after the first metal component has been incorporated, and this may be achieved in the same or an alternative manner.

After incorporation of the metal components, the molecular sieve is usually dried by heating at a temperature of 65° C. to 160° C., typically 110° C. to 143° C., for at least 1 minute and generally not longer than 24 hours, at pressures ranging from 100 to 200 kPa-a. Thereafter, the molecular sieve may be calcined in a stream of dry gas, such as air or nitrogen, at temperatures of from 260° C. to 650° C. for 1 to 20 hours. Calcination is typically conducted at pressures ranging from 100 to 300 kPa-a.

Although one advantage of the present multi-bed catalyst system is that its aromatic hydrogenation activity is low, in some cases it may be desirable to steam treat and/or sulfide one of more of the catalyst beds prior to use. Steam treatment may be effected by contacting the catalyst composition with from 5 to 100% steam at a temperature of at least 260 to 650° C. for at least one hour, typically from 1 to 20 hours, at a pressure of 100 to 2590 kPa-a. Sulfiding is conveniently accomplished by contacting the catalyst with a source of sulfur, such as hydrogen sulfide, at a temperature ranging from about 320 to 480° C. for a period of about 1 to about 24 hours.

In a particular embodiment, the catalyst of the first bed is prepared by mulling a mixture of ZSM-5, alumina binder, water, a Group 6-10 metal salt, and an additional metal salt not in Group 6-10; extruding the metal-impregnated mixture to provide an extrudate; calcining the extrudate to provide a calcined catalyst; and steaming the calcined catalyst. The calcination is performed in an environment comprising air and an inert gas, preferably nitrogen and/or argon, to a maximum environment temperature of 1000° F. (538° C.). The calcination environment may be changed from an initial composition consisting essentially of an inert gas, preferably nitrogen and/or argon, to a final composition consisting essentially of about 80% air and about 20% inert gas, by volume. The steaming is performed by heating the environment of the calcined catalyst from ambient temperature to about 750° F. (399° C.) in 100% air; increasing the temperature of the environment using steam over about a 30 min period to about 800° F. (427° C.); holding the temperature of the environment at about 800° F. (427° C.) for about 2.5 hr in 100% steam; and cooling the catalyst in air.

In another particular embodiment, the multimetallic zeolite catalyst is produced by impregnating an M/ZSM-5 extrudate with at least one metal not in Group 6-10 to provide an impregnated catalyst; and calcining the impregnated catalyst as described above.

### Transalkylation Apparatus and Process

The first and second catalyst beds and, if present, the third catalyst bed may be located in separate reactors but are conveniently located in a single reactor, typically separated from another by spacers or by inert materials, such as, alumina balls or sand. Alternatively, the first and second catalyst beds could be located in one reactor and the third catalyst bed located in a different reactor. As a further alternative, the first catalyst bed could be located in one reactor and the second and third catalyst beds located in a different reactor. In all situations, the first catalyst is not mixed with the second catalyst and the hydrocarbon feedstocks and hydrogen are arranged to contact the first catalyst bed prior to contacting the second catalyst bed. Similarly, if the third catalyst bed is present, the hydrocarbon feedstocks and hydrogen are arranged to contact the second catalyst bed prior to contacting the third catalyst bed.

In operation, the first catalyst bed is maintained under conditions effective to dealkylate aromatic hydrocarbons containing C₂+ alkyl groups in the heavy aromatic feedstock and to saturate the resulting C₂+ olefins. Suitable conditions for operation of the first catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 2 to about 20 hr⁻¹.

The second catalyst bed is maintained under conditions effective to transalkylate C9+ aromatic hydrocarbons with said at least one C₆-C₇ aromatic hydrocarbon. Suitable conditions for operation of the second catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 1 to about 10 hr⁻¹.

Where present, the third catalyst bed is maintained under conditions effective to crack non-aromatic cyclic hydrocarbons in the effluent from the second catalyst bed. Suitable conditions for operation of the third catalyst bed comprise a temperature in the range of about 100 to about 800° C., preferably about 300 to about 500° C., a pressure in the range of about 790 to about 7000 kPa-a, preferably about 2170 to 3000 kPa-a, a H₂:HC molar ratio in the range of about 0.01 to about 20, preferably about 1 to about 10, and a WHSV in the range of about 0.01 to about 100 hr⁻¹, preferably about 1 to about 50 hr⁻¹.

Obviously, where the first, second and optional third catalyst beds are located in a single reactor, the operating conditions in each bed are substantially the same.

In one embodiment, the process for the dealkylation of heavy aromatics comprises contacting a feedstream comprising C9+ aromatic hydrocarbons with a first catalyst bed comprising a M/ZSM-5 catalyst, wherein M is selected from at least one Group 6-10 metal, and at least one additional metal not in Group 6-10. The dealkylation may be carried out in the presence of C7− aromatic hydrocarbons.

In another embodiment, a feedstream comprising C9+ aromatic hydrocarbons is contacted with a first catalyst bed comprising a M/ZSM-5 catalyst, wherein M is selected from at least one Group 6-10 metal, and at least one additional metal not in Group 6-10; and then contacting the product with a second catalyst comprising a hydrogenation component and at least one crystalline zeolite effective for transalkylation, preferably including ZSM-12, in the presence of C7− aromatic hydrocarbons, to produce a transalkylation product comprising xylenes. The transalkylation product is thereafter contacted with at least one third catalyst effective for conversion of non-aromatic hydrocarbons, said third catalyst not comprising a hydrogenation component, preferably including ZSM-5.

In yet another embodiment, a feedstream comprising C9+ aromatic hydrocarbons is contacted with a first catalyst bed comprising a M/ZSM-5 catalyst, wherein M is selected from at least one Group 6-10 metal, and at least one additional metal not in Group 6-10; and then contacting the product with a second catalyst comprising a hydrogenation component and at least one crystalline zeolite effective for transalkylation, preferably including ZSM-12, in the presence of benzene, to produce a transalkylation product comprising toluene. The transalkylation product is thereafter contacted with at least one of the following catalyst systems: (i) a catalyst system effective for conversion of non-aromatic hydrocarbons, said third catalyst not comprising a hydrogenation component, preferably including ZSM-5; (ii) a catalyst system effective for transalkylation of toluene and C9+ aromatic hydrocarbons.

In order to better understand the invention, reference will be made to the following experimental work. A current state-of-the-art technology for transalkylation using a three-bed reactor system was selected to be modified according to the present invention. The current state-of-the-art technology comprises a top-bed containing a M/ZSM-5 catalyst, wherein M is a Group 6-10 metal, preferably Pt, to dealkylate ethyl and propyl side chains in a C9+ feed and saturate them by hydrogenation; the mid-bed contains a M/ZSM-12 catalyst, wherein M is a Group 6-10 metal, preferably Pt, for transalkylation; the bottom-bed contains a ZSM-5 catalyst to convert non-aromatic hydrocarbons to gas products. As would be understood by the artisan of ordinary skill, the phraseology “top-bed”, “mid-bed”, and “bottom-bed” implies a sequence of reactions from top to middle and then bottom beds. However, it will also be recognized that various feeds may be introduced at various points in the system other than just the top bed.

A variety of multimetallic catalysts were prepared either by impregnation of an existing Pt/ZSM-5 catalyst with additional metal or by mulling a mixture of ZSM-5, alumina binder, water, Pt salt, and additional metal salt followed by extrusion, calcination, and steaming The resulting top-bed catalysts were evaluated with fixed-bed micro units. The data shows that the introduction of additional metal(s) to the top-bed Pt/ZSM-5 catalyst improved catalyst selectivity toward aromatic hydrocarbon. Specifically, it reduced aromatic ring saturation when compared with Pt/ZSM-5 catalyst. This reduced C6+ non-aromatics byproducts and improved benzene purity.

The following examples are intended to illustrate the present invention and show its advantages over the prior art.

### EXAMPLE 1

Preparation of 0.115 wt % Pt/ZSM-5 catalyst by mulling, extrusion, calcination, and steaming

### Extrusion

A mixture of 649 g of ZSM-5 crystal and 825 g of Versal-300 was dry mulled for 5 minutes in a Lancaster muller. After dry mull was complete, 600 g of DI water was added to the mixture and the wet mull was done for 5 minutes. An impregnation solution made with the following composition was added to the mixture while mulling.

1.38 g of tetraamine platinum chloride

328 g of DI water

Upon addition of the Pt solution the mixture was mulled for another 10 minutes. Once complete, the metal-impregnated mixture was extruded with a 1/16″ cylinder die plate. The extrudate was dried at 250° F. (121° C.) and long extrudate was cracked into short length.

### Calcination

The extrudate was heated in flowing nitrogen (5 vol/vol/min) at 150° F./h (83.3° C./h) to 900° F. (482.2° C.), hold at 900° F. (482.2° C.) for 3 hours. While at 900° F. (482.2° C.), the gas mixture was changed to 0.25 vol/vol/min air+4.75 vol/vol/min nitrogen, hold for 30 min; 0.50 vol/vol/min air+4.50 vol/vol/min nitrogen, hold for 30 min; 1.0 vol/vol/min air+4.0 vol/vol/min nitrogen, hold for 30 min; 2.0 vol/vol/min air+3.0 vol/vol/min nitrogen, hold for 30 min. The temperature was increased at 150° F./h (83.3° C./h) to 1000° F. (538° C.). Once stabilized 1000° F. (538° C.), the gas mixture was changed to 4 vol/vol/min air+1 vol/vol/min nitrogen and hold for 6 hours. Cool down to ambient conditions and discharge.

### Steaming

The calcined catalyst was heated from ambient temperature to 750° F. (399° C.) in 100% air. The air was switched to 100% steam over a 5 min period. This switch represents t=0 for steaming Temperature was increased over a 30 minute period to 800° F. (427° C.), and then hold for an additional 2.5 hours in 100% steam. Once finished, the catalyst was cooled down in air and discharged.

### EXAMPLE 2

Preparation of Pt/Sn/ZSM-5 catalyst by impregnation of Pt/ZSM-5 with Sn salt.

### Impregnation

The catalyst described in Example 1 was used for the preparation of the 0.115 wt % Pt/0.07 wt % Sn/ZSM-5 catalyst with 1:1 Pt/Sn molar ratio. Tin (II) chloride (0.0560 g, 0.0700 wt % Sn on catalyst) was dissolved in an appropriate amount of DI water based on catalyst water absorption capacity. This solution was then sprayed on to 50 g of catalyst prepared in Example 1, while constantly rotating to ensure uniform impregnation. The wet catalyst was dried for 4 hours at 250° F. (121° C.).

### Calcination

The extrudate was introduced to an environment of 40% air and 60% nitrogen (5 vol/vol/min) The temperature was ramped from ambient to 1000° F. (538° C.) at a rate of 150° F./hr (83.3° C./h). The temperature was held for 6 hours at 1000° F. (538° C.). After the 6 hour hold, the catalyst was cooled down to ambient conditions and discharged.

### EXAMPLE 3

Preparation of Pt/Cu/ZSM-5 catalyst by impregnation of Pt/ZSM-5 with Cu salt.

The catalyst described in Example 1 was used for the preparation of the 0.115 wt % Pt/0.0375 wt % Cu/ZSM-5 catalyst with 1:1 Pt/Cu molar ratio.

### Impregnation

The catalyst described in Example 1 was used for the preparation of the 0.115 wt % Pt/0.0375 wt % Cu/ZSM-5 catalyst with 1:1 Pt/Cu molar ratio. Copper nitrate hemipentahydrate (0.0687 g, 0.0375 wt % Sn on catalyst) was dissolved in an appropriate amount of DI water based on catalyst water absorption capacity. This solution was then sprayed on to 50 g of catalyst prepared in Example 1, while constantly rotating to ensure uniform impregnation. The wet catalyst was dried for 4 hours at 250° F. (121° C.).

### Calcination

The extrudate was introduced to an environment of 40% air and 60% nitrogen (5 vol/vol/min) The temperature was ramped from ambient to 1000° F. (538° C.) at a rate of 150° F./hr (83.3° C./h). The temperature was held for 6 hours at 1000° F. (538° C.). After the 6 hour hold, the catalyst was cooled down to ambient conditions and discharged.

### EXAMPLE 4

Preparation of Pt/Ag/ZSM-5 catalyst by impregnation of Pt/ZSM-5 with Ag salt.

The catalyst described in Example 1 was used for the preparation of the 0.115 wt % Pt/0.0636 wt % Ag/ZSM-5 catalyst with 1:1 Pt/Ag molar ratio.

### Impregnation

The catalyst described in Example 1 was used for the preparation of the 0.115 wt % Pt/0.0636 wt % Ag/ZSM-5 catalyst with 1:1 Pt/Ag molar ratio. Silver (I) nitrate (0.0502 g, 0.0636 wt % Ag on catalyst) was dissolved in an appropriate amount of DI water based on catalyst water absorption capacity. This solution was then sprayed on to 50 g of catalyst prepared in Example 1, while constantly rotating to ensure uniform impregnation. The wet catalyst was dried for 4 hours at 250° F. (121° C.).

### Calcination

The extrudate was introduced to an environment of 40% air and 60% nitrogen (5 vol/vol/min) The temperature was ramped from ambient to 1000° F. (538° C.) at a rate of 150° F./hr (83.3° C./h). The temperature was held for 6 hours at 1000° F. (538° C.). After the 6 hour hold, the catalyst was cooled down to ambient conditions and discharged.

### EXAMPLE 5

Preparation of Pt/Ca/ZSM-5 catalyst by impregnation of Pt/ZSM-5 with Cu salt.

The catalyst described in Example 1 was used for the preparation of the 0.115 wt % Pt/0.0236 wt % Ca/ZSM-5 catalyst with 1:1 Pt/Ca molar ratio.

### Impregnation

The catalyst described in Example 1 was used for the preparation of the 0.115 wt % Ca/0.0236 wt % Ca/ZSM-5 catalyst with 1:1 Pt/Ca molar ratio. Calcium nitrate (0.0168 g, 0.0236 wt % Ca on catalyst) was dissolved in an appropriate amount of DI water based on catalyst water absorption capacity. This solution was then sprayed on to 50 g of catalyst prepared in Example 1, while constantly rotating to ensure uniform impregnation. The wet catalyst was dried for 4 hours at 250° F. (121° C.).

### Calcination

The extrudate was introduced to an environment of 40% air and 60% nitrogen (5 vol/vol/min) The temperature was ramped from ambient to 1000° F. (538° C.) at a rate of 150° F./hr (83.3° C./h). The temperature was held for 6 hours at 1000° F. (538° C.). After the 6 hour hold, the catalyst was cooled down to ambient conditions and discharged.

### EXAMPLE 6

Preparation of Pt/Mg/ZSM-5 catalyst by impregnation of Pt/ZSM-5 with Mg salt.

The catalyst described in Example 1 was used for the preparation of the 0.115 wt % Pt/0.0145 wt % Mg/ZSM-5 catalyst with 1:1 Pt/Mg molar ratio.

### Impregnation

The catalyst described in Example 1 was used for the preparation of the 0.115 wt % Pt/0.0145 wt % Mg/ZSM-5 catalyst with 1:1 Pt/Mg molar ratio. Magnesium nitrate (0.0123 g, 0.0145 wt % Mg on catalyst) was dissolved in an appropriate amount of DI water based on catalyst water absorption capacity. This solution was then sprayed on to 50 g of catalyst prepared in Example 1, while constantly rotating to ensure uniform impregnation. The wet catalyst was dried for 4 hours at 250° F. (121° C.).

### Calcination

The extrudate was introduced to an environment of 40% air and 60% nitrogen (5 vol/vol/min) The temperature was ramped from ambient to 1000° F. (538° C.) at a rate of 150° F./hr (83.3° C./h). The temperature was held for 6 hours at 1000° F. (538° C.). After the 6 hour hold, the catalyst was cooled down to ambient conditions and discharged.

### EXAMPLE 7

Preparation of Pt/Cu/ZSM-5 catalyst by mulling, extrusion, calcination, and steaming.

### Extrusion

A mixture of 225 g of ZSM-5 crystal and 284.38 g of Versal-300 was dry mulled for 5 minutes in a Lancaster muller. After dry mull was complete, 187.80 g of DI water was added to the mixture and the wet mull was done for 5 minutes. An impregnation solution made with the following composition was added to the mixture while mulling.

13.265 g of tetraamine platinum nitrate (an aqueous solution with 3.55 wt % Pt)

0.561 g of copper (II) nitrate hemipentahydrate

108 g of DI water

Upon addition of the Pt/Cu solution, the mixture was mulled for another 10 minutes. Once complete, the metal-impregnated mixture was extruded with a 1/16″ cylinder die plate. The extrudate was dried at 250° F. (121° C.) and long extrudate was cracked into short length.

### Calcination

The extrudate was heated in flowing nitrogen (5 vol/vol/min) at 150° F./h (83.3° C./h) to 900° F. (482.2° C.), hold at 900° F. (482.2° C.) for 3 hours. While at 900° F. (482.2° C.), the gas mixture was changed to 0.25 vol/vol/min air+4.75 vol/vol/min nitrogen, hold for 30 min; 0.50 vol/vol/min air+4.50 vol/vol/min nitrogen, hold for 30 min; 1.0 vol/vol/min air+4.0 vol/vol/min nitrogen, hold for 30 min; 2.0 vol/vol/min air+3.0 vol/vol/min nitrogen, hold for 30 min. The temperature was increased to 1000° F. (538° C.) at a rate of 150° F./hr (83.3° C./h). Once stabilized 1000° F. (538° C.), the gas mixture was changed to 4 vol/vol/min air+1 vol/vol/min nitrogen and hold for 6 hours. Cool down to ambient conditions and discharge.

### Steaming

The calcined catalyst was heated from ambient temperature to 750° F. (399° C.) in 100% air. The air was switched to 100% steam over a 5 min period. This switch represents t=0 for steaming Temperature was increased over a 30 minute period to 800° F. (427° C.), and then hold for an additional 2.5 hours in 100% steam. Once finished, the catalyst was cooled down in air and discharged.

### EXAMPLE 8

Preparation of Pt/Sn/ZSM-5 catalyst by mulling, extrusion, calcination, and steaming

### Extrusion

A mixture of 649 g of ZSM-5 crystal and 825 g of Versal-300 was dry mulled for 5 minutes in a Lancaster muller. After dry mull was complete, 600 g of DI water was added to the mixture and the wet mull was done for 5 minutes. An impregnation solution made with the following composition was added to the mixture while mulling.

1.38 g of tetraamine platinum chloride

1.34 g of tin chloride

328 g of DI water

Upon addition of the Pt/Sn solution, the mixture was mulled for another 10 minutes. Once complete, the metal-impregnated mixture was extruded with a 1/16″ cylinder die plate. The extrudate was dried at 250° F. (121° C.) and long extrudate was cracked into short length.

### Calcination

The extrudate was heated in flowing nitrogen (5 vol/vol/min) at 150° F./h (83.3° C./h) to 900° F. (482.2° C.), hold at 900° F. (482.2° C.) for 3 hours. While at 900° F. (482.2° C.), the gas mixture was changed to 0.25 vol/vol/min air + 4.75 vol/vol/min nitrogen, hold for 30 min; 0.50 vol/vol/min air+4.50 vol/vol/min nitrogen, hold for 30 min; 1.0 vol/vol/min air+4.0 vol/vol/min nitrogen, hold for 30 min; 2.0 vol/vol/min air+3.0 vol/vol/min nitrogen, hold for 30 min. The temperature was increased to 1000° F. (538° C.) at a rate of 150 F/hr (83.3° C./h). Once stabilized 1000° F. (538° C.), the gas mixture was changed to 4 vol/vol/min air+1 vol/vol/min nitrogen and hold for 6 hours. Cool down to ambient conditions and discharge.

### Steaming

The calcined catalyst was heated from ambient temperature to 750° F. (399° C.) in 100% air. The air was switched to 100% steam over a 5 min period. This switch represents t=0 for steaming Temperature was increased over a 30 minute period to 800° F. (427° C.), and then hold for an additional 2.5 hours in 100% steam. Once finished, the catalyst was cooled down in air and discharged.

### EXAMPLE 9

### Catalyst Evaluation

A fixed bed reactor with ⅜″ external diameter was used for the evaluation. The reactor was equipped with a ⅛″ diameter thermal well to monitor reactor temperature at the center of the catalyst bed. One gram of catalyst made in Example 1 in the shape of cylindrical 1/16″ extrudate was loaded to the reactor.

The reactor pressure was set at 350 psig with a steady flow of H₂ at 76 cc/min. The reactor temperature was increased at 10° C./min to 400° C., and held at 400° C. for 1 hour. Temperature was then increased to 430° C. When temperature and gas flow are steady, feed (see composition in Table 1) was introduced at 11.46 cc/hr or 10 WHSV (feed density 0.872 g/cc). Once feed was introduced to the reactor, the unit was held at the set conditions for 12 hours to fully de-edge the catalyst. At the end of the de-edging, the reactor temperature was reduced to 380° C. and H₂ flow was increased to 76 cc/min for catalyst activity test. Catalyst performance was measured at 380, 390, 400, and 410° C. for 12 hours each while the run condition was held at 350 psig, 10 WHSV, and 2:1 H₂/HC ratio. The reactor temperature was ramped down at −5° C./hr to 380° C. Once the temperature reached 380° C. and stabilized, the H₂ flow was increased to 114 cc/min and feed flow was increased to 17.2 cc/hr (15 WHSV). Catalyst performance was further measured at 380, 390, 400, and 410° C. for 12 hours each while the run condition was held at 350 psig, 15 WHSV, and 2:1 H₂/HC ratio. At each temperature including the de-edging, 3 GC shots were taken at 4 hr intervals by an online GC equipped with a DB-1 and a DB-Wax column.

The rest of the catalysts made in Examples 2 to 7 were also evaluated using the same procedure described above. The results are compared in the Discussions Section below.

TABLE 1

| Component | wt % |
|---|---|
| benzene | 8.6 |
| toluene | 7.2 |
| ethylbenzene | <0.1 |
| o-xylene | 0.1 |
| m-xylene | <0.1 |
| p-xylene | <0.1 |
| C9 aromatics | 62.1 |
| C10 aromatics | 21.6 |
| C11 aromatics | 0.2 |
| Total | 100.0 |

### Results and Discussions

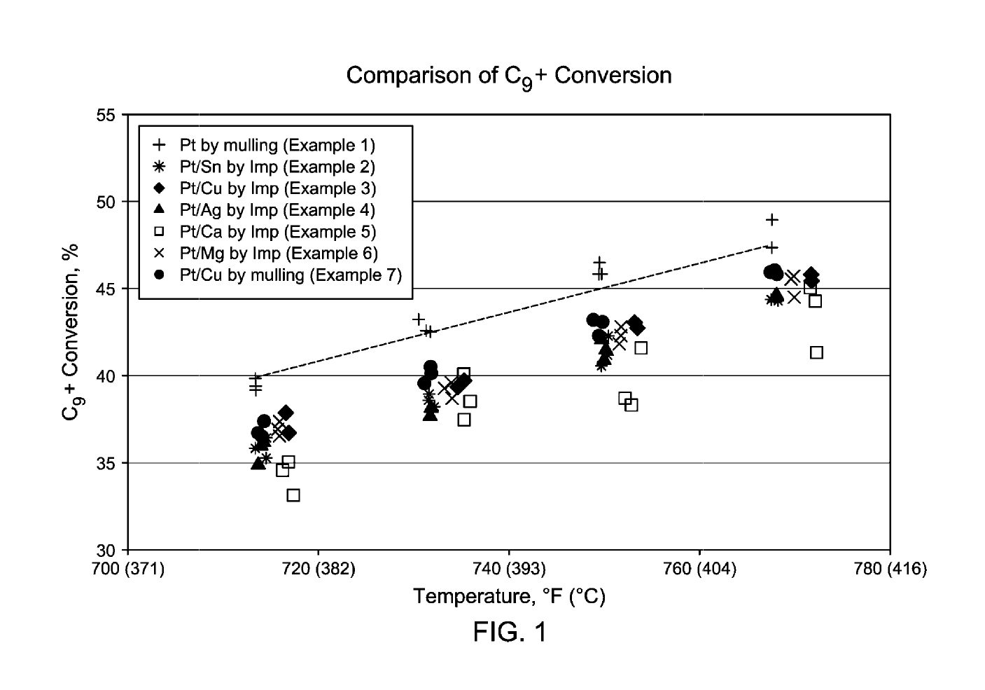

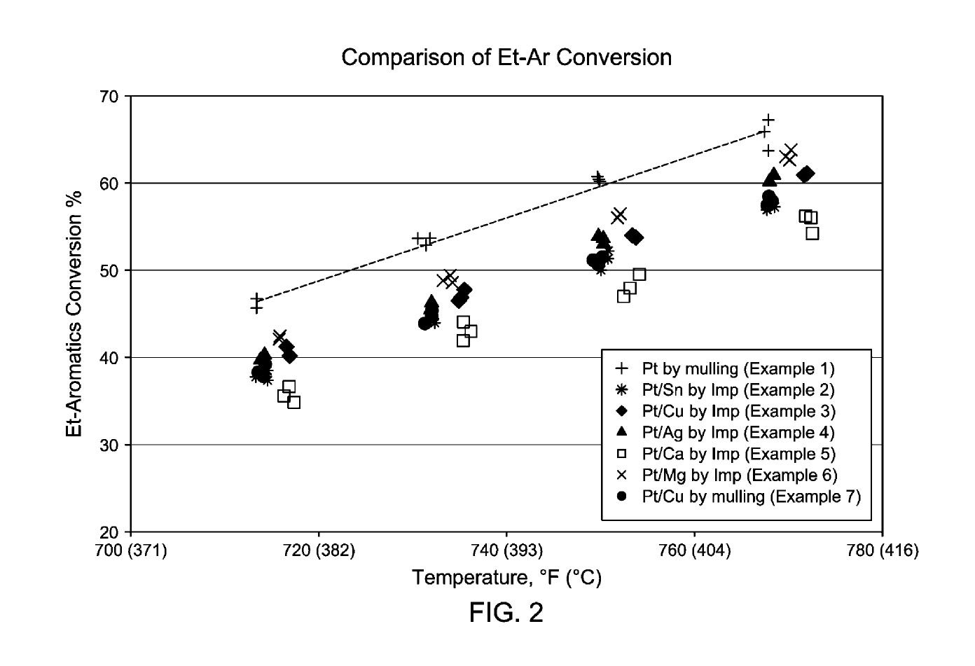

FIGS. 1 and 2 compare catalyst activity for C9+ and ethyl-aromatics conversion. The bimetallic catalysts were less active than the Pt catalysts made in Example 1: the bimetallic catalysts provided a lower conversion at a given temperature; they required a higher temperature to achieve a constant conversion.

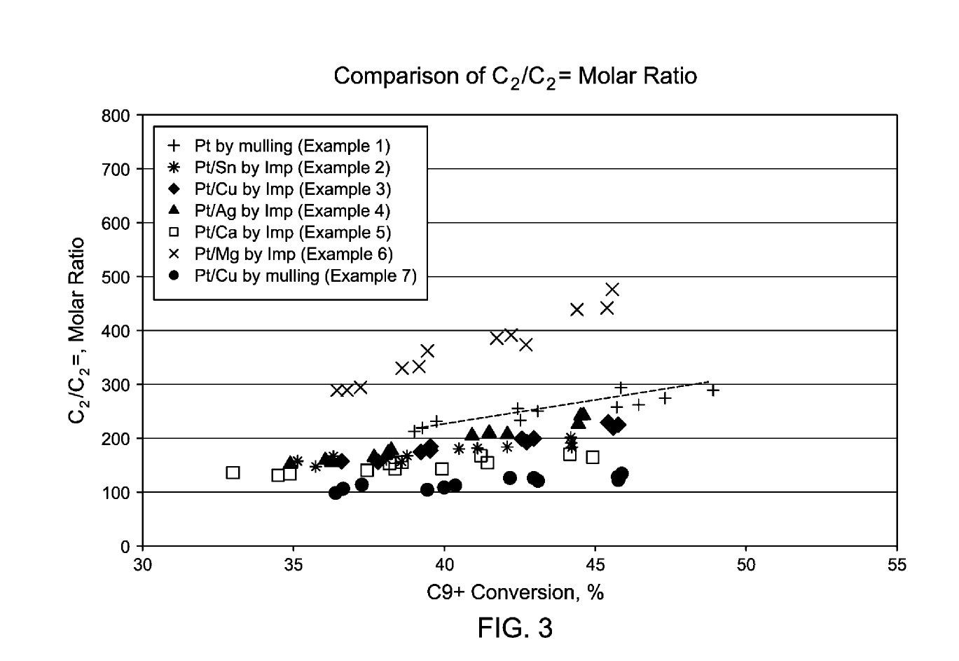

FIG. 3 shows that, except for the Pt/Mg catalyst, the bimetallic catalysts were slightly less selective than the Pt catalysts to saturate ethylene (C₂: ethane, C₂⁼ ethylene).

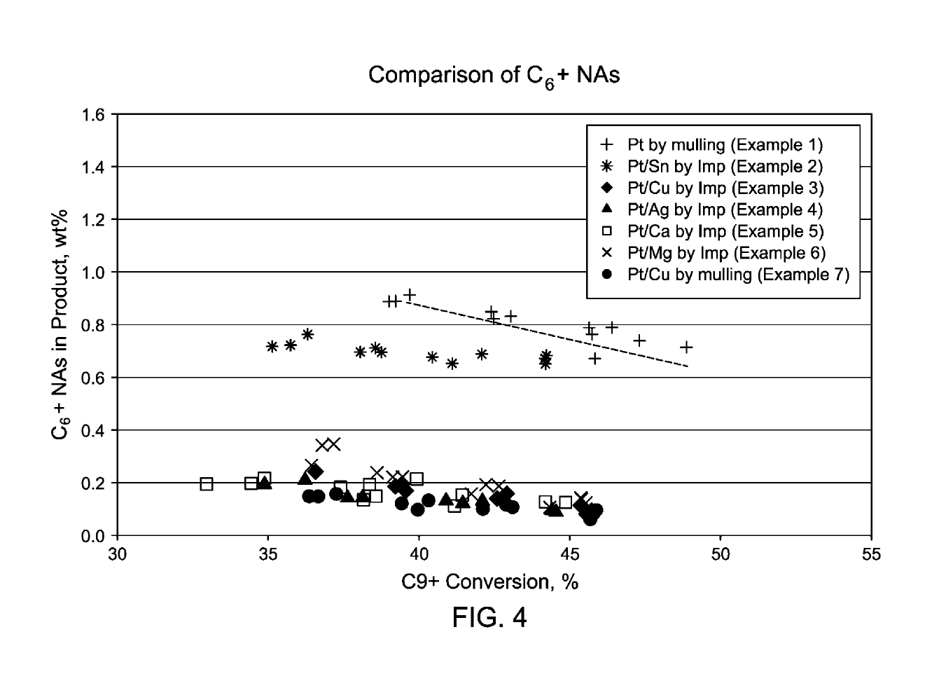

FIG. 4 shows that by impregnating the Pt catalyst (Example 1) with a second metal, the catalysts (Examples 2 to 6) had significant reduction in C6+ NAs (non-aromatics). The same is true for catalyst made by mulling as in Example 7: the Pt/Cu catalyst had significant reduction in C6+ NAs when compared with the Pt catalyst.

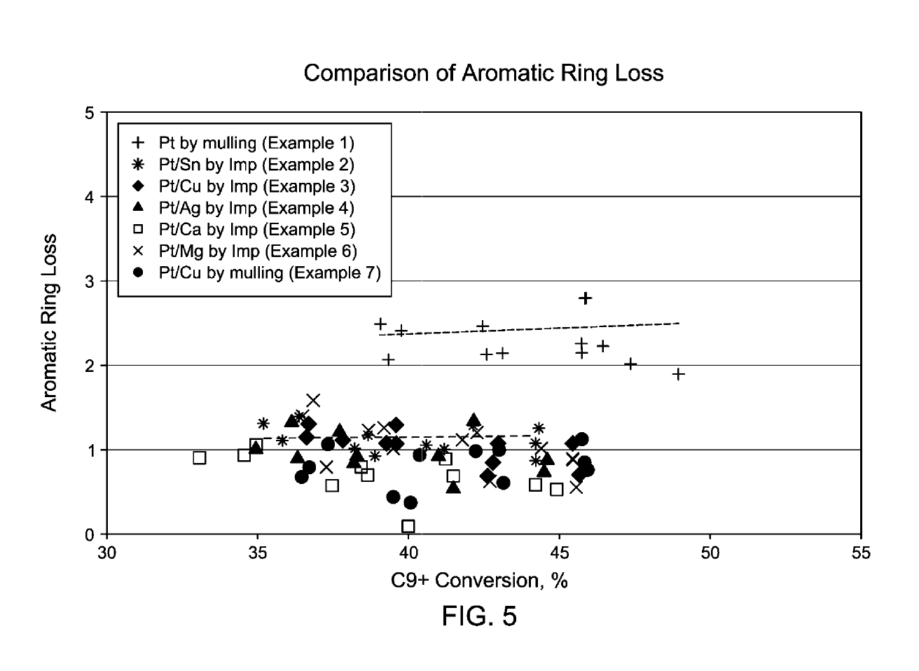

The most significant feature of the bimetallic and multi-metallic catalysts are shown in FIG. 5: the bimetallic catalysts significantly reduced aromatic ring saturation when compared with the Pt catalyst. Because of this feature, the benzene purity was improved as well.

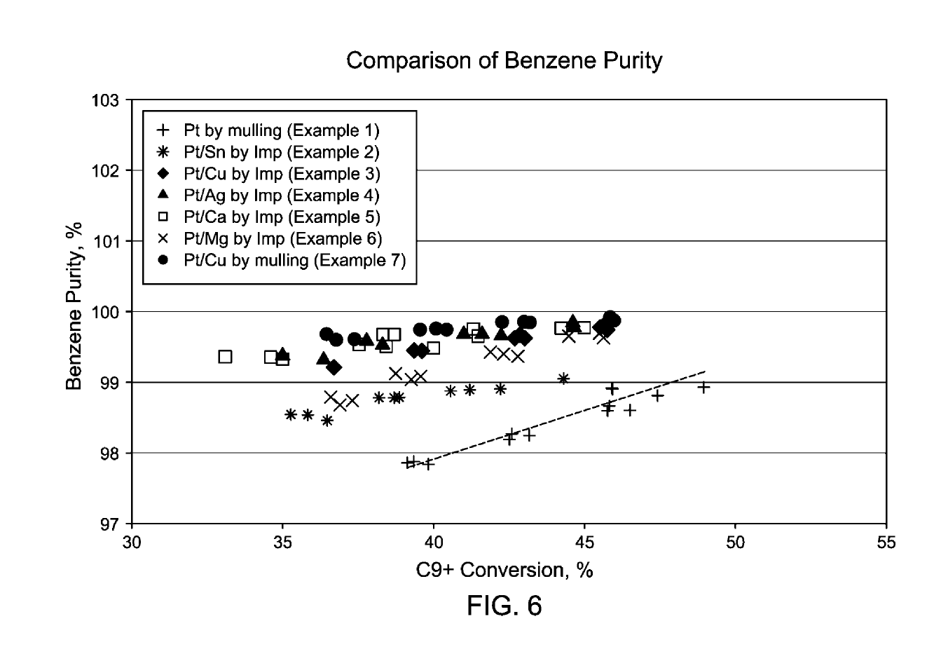

FIG. 6: All the bimetallic catalysts (Examples 2 to 7) had higher benzene purity than their parent Pt catalyst made in Example 1.

The invention has been described above with reference to numerous embodiments and specific examples. Many variations will suggest themselves to those skilled in this art in light of the above detailed description. All such obvious variations are within the full intended scope of the appended claims.

Trade names used herein are indicated by a ™ symbol or ® symbol, indicating that the names may be protected by certain trademark rights, e.g., they may be registered trademarks in various jurisdictions. All patents and patent applications, test procedures (such as ASTM methods, UL methods, and the like), and other documents cited herein are fully incorporated by reference to the extent such disclosure is not inconsistent with this invention and for all jurisdictions in which such incorporation is permitted. When numerical lower limits and numerical upper limits are listed herein, ranges from any lower limit to any upper limit are contemplated.

What is claimed is:

1. A reactor system comprising:
a) a first bed comprising a multimetallic zeolite catalyst, said multimetallic zeolite catalyst comprising:
i) an M/ZSM-5 catalyst, wherein M is selected from at least one Group 6-10 metal; and
ii) at least one additional metal not in Group 6-10; and
b) a second bed, downstream of said first bed, comprising a second catalyst comprising a hydrogenation component and a crystalline zeolite.

2. The reactor system of claim 1, wherein the system contains a third bed located down-stream of the second bed comprising a third catalyst without a hydrogenation component and which is suitable to crack non-aromatic hydrocarbon species.

3. The reactor system of claim 1, wherein M is platinum.

4. The reactor system of claim 1, wherein the additional metal is selected from the group consisting of tin, copper, silver, calcium, and magnesium.

5. The reactor system of claim 1, wherein M is present in the amount of 0.110% and 0.120% by weight, based on the weight of said M/ZSM-5 catalyst.

6. The reactor system of claim 1, wherein M and the additional metal not in Group 6-10 have a molar ratio between 0.7:1 and 1.3:1.

7. The reactor system of claim 1, wherein M and the additional metal not in Group 6-10 have a molar ratio of about 1:1.

8. The reactor system of claim 1, wherein said multimetallic zeolite catalyst is produced by a process comprising:
a) mulling a mixture of ZSM-5, alumina binder, water, a Group 6-10 metal salt, and an additional metal salt not in Group 6-10;
b) extruding the metal-impregnated mixture to provide an extrudate;
c) calcining the extrudate to provide a calcined catalyst; and
d) steaming the calcined catalyst.

9. The reactor system of claim 8, wherein the extrudate is calcined in an environment comprising air and an inert gas, preferably nitrogen and/or argon, to a maximum environment temperature of 1000° F. (538° C.).

10. The reactor system of claim 9, wherein the environment is changed from an initial composition consisting essentially of an inert gas, preferably nitrogen and/or argon, to a final composition consisting essentially of about 80% air and about 20% inert gas, by volume.

11. The reactor system of claim 8, wherein the steaming in step d) comprises:
a) heating the environment of the calcined catalyst from ambient temperature to about 750° F. (399° C.) in 100% air;
b) increasing the temperature of the environment using steam over about a 30 min period to about 800° F. (427° C.);
c) holding the temperature of the environment at about 800° F. (427° C.) for about 2.5 hr in 100% steam; and
d) cooling the catalyst in air.

12. The reactor system of claim 1, wherein the multimetallic zeolite catalyst is produced by a process comprising:
a) impregnating an M/ZSM-5 extrudate with at least one metal not in Group 6-10 to provide an impregnated catalyst; and
b) calcining the impregnated catalyst.

13. The reactor system of claim 12, wherein the impregnated catalyst is calcined in an environment comprising air and nitrogen to a maximum temperature of 1000° F. (538° C.).

14. The reactor system of claim 2, wherein the second catalyst comprises ZSM-12 and the third catalyst comprises ZSM-5.

15. A process for the dealkylation of heavy aromatics comprising:
contacting a feedstream comprising C9+ aromatic hydrocarbons with a first catalyst bed comprising a multimetallic zeolite catalyst, said multimetallic zeolite catalyst comprising:
i) an M/ZSM-5 catalyst, wherein M is selected from at least one Group 6-10 metal; and
ii) at least one additional metal not in Group 6-10.

16. The process of claim 15, wherein the dealkylation is carried out in the presence of C7− aromatic hydrocarbons.

17. A process for producing p-xylene comprising:
a) contacting a feedstream comprising C9+ aromatic hydrocarbons with a first catalyst bed comprising a multimetallic zeolite catalyst, said multimetallic zeolite catalyst comprising:
i) an M/ZSM-5 catalyst, wherein M is selected from at least one Group 6-10 metal; and
ii) at least one additional metal not in Group 6-10; and then
b) contacting the product of a) with a second catalyst comprising a hydrogenation component and at least one crystalline zeolite effective for transalkylation in the presence of C7− aromatic hydrocarbons, to produce a transalkylation product comprising xylenes.

18. The process of claim 17, wherein the transalkylation product of b) is thereafter contacted with at least one third catalyst effective for conversion of non-aromatic hydrocarbons, said third catalyst not comprising a hydrogenation component.

19. The process of claim 18, wherein the second catalyst comprises ZSM-12 and the third catalyst comprises ZSM-5.

20. A process comprising:
a) contacting a feedstream comprising C9+ aromatic hydrocarbons with a first catalyst bed comprising a multimetallic zeolite catalyst, said multimetallic zeolite catalyst comprising:
i) an M/ZSM-5 catalyst, wherein M is selected from at least one Group 6-10 metal; and
ii) at least one additional metal not in Group 6-10; and then
b) contacting the product of a) with a second catalyst comprising a hydrogenation component and at least one crystalline zeolite effective for transalkylation in the presence of benzene, to produce a transalkylation product comprising toluene.

21. The process of claim 20, wherein the transalkylation product of b) is thereafter contacted with at least one of the following catalyst systems: (i) a catalyst system effective for conversion of non-aromatic hydrocarbons, said third catalyst not comprising a hydrogenation component; and (ii) a catalyst system effective for transalkylation of toluene and C9+ aromatic hydrocarbons.

22. The process of claim 21, wherein the second catalyst comprises ZSM-12 and the third is catalyst comprises ZSM-5.
